(19) United States
(12) Patent Application Publication
(10) Pub. No.: US 2007/0281314 A1
(43) Pub. Date: Dec. 6, 2007

(54) Methods for capture and detection of micro-RNA molecules from the skin by non-invasive tape stripping

(75) Inventor: Nicholas R. Benson, San Diego, CA (US)

Correspondence Address: DLA PIPER US LLP, 4365 EXECUTIVE DRIVE, SUITE 1100, SAN DIEGO, CA, 92121-2133, US

(73) Assignee: DERMTECH INTERNATIONAL, San Diego, CA (US)

(21) Appl. No.: 11/788,644

(22) Filed: Apr. 20, 2007

Related U.S. Application Data
(60) Provisional application No. 60/794,705, filed on Apr. 24, 2006.
(60) Provisional application No. 60/794,057, filed on Apr. 20, 2006.

(51) Int. Cl. C12Q 1/68; C07H 21/02

(52) U.S. Cl. 435/6; 536/23.1

(57) ABSTRACT

The present invention provides non-invasive methods for isolating an miRNA molecule from an epidermal sample, as well as detecting, monitoring, and diagnosing skin disease and pathological skin states such as irritated skin and psoriasis. The methods include using tape stripping to obtain the miRNA from epidermal samples, and may be used to further analyze expression of one or more skin markers. In illustrative examples, the tape stripping is performed using pliable tape that has a rubber adhesive. Furthermore, the present invention provides methods for predicting and monitoring response to therapy for a skin disease, such as psoriasis or dermatitis. Finally, the methods can include the use of a microarray.

### RELATED APPLICATION DATA

This application claims priority under 35 U.S.C. §119(e) to U.S. Provisional Patent Application No. 60/794,705, filed Apr. 24, 2006, and No. 60/794,057, filed Apr. 20, 2006, each of which is herein incorporated by reference in its entirety.

### BACKGROUND OF THE INVENTION

1. Field of the Invention

The present invention relates generally to non-invasive diagnostics methods and more specifically to methods for isolating and analyzing nucleic acids from skin samples.

2. Background Information

Skin diseases represent major health care challenges today. For example, over five million Americans and over one hundred million people worldwide suffer from psoriasis. Detection, diagnosis, and staging of a skin disease are important aspects of its management. Current diagnostic methods rely mainly on visible observation and biopsies. However, detection methods for skin diseases that rely on visible observation are not effective for diagnosing many skin diseases, and do not detect diseases until after clinical manifestation. Invasive methods such as biopsies, not only are traumatic for a subject being tested, they also increase the risk of infection. Furthermore, invasive methods do not provide an enriched sample of cells on the surface of skin, which are the cells involved in a surface reaction.

Detection and diagnosis of skin disease are important not only for patient management, but also to assess the safety and efficacy of new skin disease therapeutic agents and new skin care products. New therapeutic agents are required for many skin diseases where present therapeutic agents are not fully effective. Furthermore, diagnostic methods provide important information regarding the specific genetic changes underlying a subject's skin disease. Identifying these genetic changes identifies potential drug targets and may be critical in determining whether a person will respond to a particular therapeutic agent.

In addition to assessing new therapeutic agents, detection and diagnosis methods are also important to assess the safety of new skin care products. Skin care products, including cosmetics, are an important part of most people's daily grooming habits. The average adult uses at least seven different skin care products each day. Currently, all commercial skin care products are required to undergo safety testing. These tests take the form of Clinical Acute Primary Irritation and 14-day Cumulative Irritation Protocols followed by Human Repeat Insult Patch Testing (HRIPT) to detect sensitization (contact allergy). Visual analysis is used to determine the test results. Therefore, allergic reactions are not detected until they have manifested themselves in a visible reaction.

### SUMMARY OF THE INVENTION

The present invention is based on a non-invasive approach for recovering nucleic acids such as DNA, messenger RNA, or micro-RNA (miRNA or proteins) from the surface of skin via a simple tape stripping procedure that permits a direct quantitative and qualitative assessment of biomarkers. The method provides valuable genetic information, not obtainable using a visible detection method. Furthermore, although tape-harvested RNA is shown to be comparable in quality and utility to RNA recovered by biopsy, the method provides information regarding cells of the outermost layers of the skin that is not obtained using biopsy samples. Finally, the method is far less traumatic than a biopsy.

Provided herein is a method for isolating a miRNA molecule from an epidermal sample from skin, including applying an adhesive tape to a target area of the skin in a manner sufficient to isolate an epidermal sample adhering to the adhesive tape, wherein the epidermal sample comprises miRNA molecules. In one embodiment, the tape includes a rubber-based adhesive and is pliable.

Other embodiments are based in part on the discovery that for tape stripping of the skin, non-polar, pliable, adhesive tapes, especially pliable tapes with rubber adhesive, are more effective than other types of adhesive tapes. Using pliable tapes with rubber adhesives, as few as 10 or less tape strippings and in certain examples as few as 4 or even 1 tape stripping can be used to isolate nucleic acid molecules such as miRNA, or proteins from the epidermal layer of the skin.

In another embodiment, the invention provides a method for identifying an miRNA profile indicative of a disease, pathological or physiological state of a human subject, the method by applying an adhesive tape to an area of skin afflicted with the disease, pathological or physiological state in a manner sufficient to isolate an epidermal sample adhering to the adhesive tape, wherein the epidermal sample comprises miRNA molecules. The method includes determining miRNA levels from the sample, or an amplification product thereof, and comparing the levels with the miRNA levels of an unaffected sample, wherein a difference in miRNA level is indicative of an miRNA profile of a disease, pathological or physiological state of a subject. The disease, pathological or physiological state may be any disease associated with the skin such as, dermatitis, psoriasis and diabetes. The disease, pathological or physiological state may be any cancer such as, melanoma, carcinoma, and prostate cancer. In another embodiment, the method further includes comparing the miRNA levels to expression levels of mRNA isolated from the same sample, thereby expression profiling the mRNA population in the sample.

In another embodiment, the invention provides a method for predicting response to treatment for a disease, pathological or physiological state, comprising: a) applying an adhesive tape to an area of skin in a manner sufficient to isolate an epidermal sample adhering to the adhesive tape, wherein the epidermal sample comprises miRNA molecules; and b) determining miRNA levels from the sample, or an amplification product thereof, and comparing the levels with the miRNA levels of an unaffected subject or sample, wherein a difference in miRNA level is indicative of a response to treatment of a disease, pathological or physiological state. In this method, the subject may have a disease, such as diabetes, that is not readily apparent from a particular area of the skin.

In another embodiment the invention provides a kit for isolation and recovery of a miRNA molecule from an epidermal sample. The kit includes an adhesive tape for performing methods provided herein. Accordingly, in one embodiment, provided herein is a kit that contains a pliable adhesive tape made up at least in part, of a non-polar polymer. In certain aspects, the tape is a rubber-based tape.

### DETAILED DESCRIPTION OF THE INVENTION

The present invention is based on a non-invasive approach for recovering or analyzing nucleic acids such as DNA or RNA, or proteins from the surface of skin via a simple tape stripping procedure that permits a direct quantitative and qualitative assessment of pathologic and physiologic biomarkers. Tape-harvested RNA is shown to be comparable in quality and utility to RNA recovered by biopsy. The present method causes little or no discomfort to the patient. Therefore, it can be performed routinely in a physician's office, for example, for point of care testing. Accordingly, provided herein are methods and markers for non-invasive isolation and/or recovery of nucleic acids, such as miRNA, from epidermal samples using tape stripping.

Tape stripping is a widely employed method in experimental dermatology. Uses for the technique include: studying the role of the stratum comeum (SC) in barrier function, in vivo, (van der Valk and Maibach 1990; Kalia, Pirot et al. 1996; Bashir, Chew et al. 2001), as a non-invasive method of recovering SC for assessing percutaneous absorption (Rougier, Dupuis et al. 1983; Rougier, Dupuis et al. 1985; Cullander, Jeske et al. 2000; Bunge and Guy 2003); and the stimulation of injury and irritation without the use of chemicals (Gerritsen, van Erp et al. 1994; Nickoloff and Naidu 1994; Marionnet, Bernerd et al. 2003).

The use of tape stripping as an analytical or quantitative tool for assessing percutaneous absorption was initially promising (Rougier, Dupuis et al. 1986) but the uneven nature of the SC and variable removal of SC with each tape strip created an uncertainty in the method that led to doubt as to its potential as a reliable and quantitative technique (Marttin, Neelissen-Subnel et al. 1996; van der Molen, Spies et al. 1997; Bunge and Guy 2003). Most of the deficiencies in the use of tape stripping as a pharmacokinetic tool were grounded in the difficulty in defining an appropriate method of normalizing data that made drug measurements independent of the absolute mass of stratum corneum recovered with tape.

Morhenn et al. (Morhenn, Chang et al. 1999) made an important innovation to the tape stripping method when they showed that RNA could be recovered from cells adherent to the tape. The authors showed that RNA recovered from normal, sodium laurel sulfate-irritated (SLS; simulating irritant contact dermatitis) and nickel sulfate treated (simulating allergic contact dermatitis) skin sites could be differentiated based on expression of the IL-4 and IL-8 mRNAs. Expression of these mRNAs in each sample was normalized to GAPDH, thus accounting for the variable recovery of RNA in individual samples.

Wong et al. (Wong, Tran et al. 2004) further improved tape stripping by reducing the number of tapes required to strip a site from greater than 20 to 4, and demonstrating that mRNA recovered could be quantified by RT-PCR and profiled by DNA microarrays. In addition, by characterizing tape strip recovered mRNA from SLS-treated, water-treated and normal skin by microarray, Wong et al. revealed hundreds of novel, differentially expressed mRNAs involved in the response to SLS-irritation. The utility of the improved technique was highlighted by the fact that of the 100 most differentially expressed mRNAs (p<10⁻⁹), 60% were previously identified in the literature as being involved in inflammation and wound healing, while the remaining genes were novel to contact irritant dermatitis. Thus it was clearly shown that mRNA recovered by tape stripping could accurately convey the real time physiology of the skin and could be used to derive differential expression profiles of affected skin.

“Nucleic acid” means DNA, RNA, single-stranded, double-stranded or triple stranded and any chemical modifications thereof. Virtually any modification of the nucleic acid is contemplated. A “nucleic acid” can be of almost any length, from 10, 20, 30, 40, 50, 60, 75, 100, 125,150, 175, 200, 225, 250, 275, 300, 400, 500, 600, 700, 800, 900, 1000, 1500, 2000, 2500, 3000, 3500, 4000, 4500, 5000, 6000, 7000, 8000, 9000, 10,000, 15,000, 20,000, 30,000, 40,000, 50,000, 75,000, 100,000, 150,000, 200,000, 500,000, 1,000,000, 1,500,000, 2,000,000, 5,000,000 or even more bases in length, up to a full-length chromosomal DNA molecule. For methods that analyze expression of a gene, the nucleic acid isolated from a sample is typically RNA.

Micro-RNAs (miRNA) are small single stranded RNA molecules an average of 22 nucleotides long that are involved in regulating mRNA expression in diverse species including humans (reviewed in Bartel 2004). The first report of miRNA was that of the lin-4 gene, discovered in the worm C. elegans (Lee, Feinbaum et al. 1993). Since then hundreds of miRNAs have been discovered in flies, plants and mammals.

miRNAs regulate gene expression by binding to the 3′-untranslated regions of mRNA and catalyze either i) cleavage of the mRNA; or 2) repression of translation. The regulation of gene expression by miRNAs is central to many biological processes such as cell development, differentiation, communication, and apoptosis (Reinhart, Slack et al. 2000; Baehrecke 2003; Brennecke, Hipfner et al. 2003; Chen, Li et al. 2004). Recently it has been shown that miRNA are active during embryogenesis of the mouse epithelium and play a significant role in skin morphogenesis (Yi, O'Carroll et al. 2006).

Given the role of miRNA in gene expression it is clear that miRNAs will influence, if not completely specify the relative amounts of mRNA in particular cell types and thus determine a particular gene expression profile (i.e. a population of specific mRNAs) in different cell types. In addition, it is likely that the particular distribution of specific miRNAs in a cell will also be distinctive in different cell types. Thus, determination of the miRNA profile of a tissue may be as valuable a tool as expression profiling the actual mRNA population in that tissue.

Accordingly, miRNA levels and/or detection of miRNA mutations are useful for the purposes of disease detection, diagnosis, prognosis, or treatment-related decisions (i.e. indicate response either before or after drug has been administered) or detection and prognosis of virus infection. Such measurements may additionally be used to discern specific signal transduction pathways involved in development of cutaneous or systemic states, including disease or conditions (e.g. types of baldness, allergic vs. irritant skin reaction, etc.), for purposes of drug discovery and development.

To date, the current application of tape stripping the skin revolves around the recovery of mRNA from the cells adherent to the tape. There has been no determination of the ability to recover miRNA using the tape stripping method. In this report we demonstrate the recovery of microRNA from the surface of the skin with the use of adhesive tape for the first time.

The term “sample” refers to any preparation derived from skin of a subject. For example, a sample of cells obtained using the non-invasive method described above can be used to isolate polynucleotides, polypeptides, or lipids, preferably polynucleotides and polypeptides, most preferably nucleic acid molecules, such as polynucleotides, for the methods of the present invention. Samples for the present invention, typically are taken from a skin lesion, that is suspected of being the result of a disease or a pathological or physiological state, such as psoriasis or dermatitis. The samples are taken of the skin surface of the suspicious lesion using non-invasive skin sampling methods discussed herein.

The term “normal sample” or “control sample” refers to any sample taken from a subject of similar species that is considered healthy or otherwise not suffering from a particular disease, pathological or physiological state. As such, a normal/standard level of miRNA denotes the level of miRNA present in a sample from the normal sample. A normal level of miRNA can be established by combining skin samples or cell extracts taken from normal healthy subjects and determining the level of miRNA present. In addition, a normal level of miRNA also can be determined as an average value taken from a population of subjects that is considered to be healthy, or is at least free of a particular disease, pathological or physiological state. Accordingly, levels of miRNA in subject, control, and disease samples can be compared with the standard values. Deviation between standard and subject values establishes the parameters for diagnosing disease.

The term “skin” refers to the outer protective covering of the body, consisting of the epidermis (including the stratum corneum) and the underlying dermis, and is understood to include sweat and sebaceous glands, as well as hair follicle structures. Throughout the present application, the adjective “cutaneous” can be used, and should be understood to refer generally to attributes of the skin, as appropriate to the context in which they are used. In a preferred embodiment, the skin is mammalian skin. In an illustrative embodiment the skin is human skin.

The tape stripping methods provided herein typically involve applying an adhesive tape to the skin of a subject and removing the adhesive tape from the skin of the subject one or more times. In certain examples, the adhesive tape is applied to the skin and removed from the skin about one to ten times. Alternatively, about ten adhesive tapes can be sequentially applied to the skin and removed from the skin. These adhesive tapes are then combined for further analysis. Accordingly, an adhesive tape can be applied to and removed from a target site 10, 9, 8, 7, 6, 5, 4, 3, 2, or 1 time, and/or 10, 9, 8, 7, 6, 5, 4, 3, 2, or 1 adhesive tape can be applied to and removed from the target site. In one illustrative example, the adhesive tape is applied to the skin between about one and eight times, in another example, between one and five times, and in another illustrative example the tape is applied and removed from the skin four times.

For the tape strippings, the same strip of tape can be repeatedly applied to, and removed from, a target site. However, in illustrative embodiments a fresh piece of adhesive tape is sequentially applied to a target site of the skin. The individual tape strips used to sample a site can then be combined into one extraction vessel for further processing such as nucleic acid extraction. In one illustrative example, the adhesive tape is applied to the skin between about one and eight times, in another example, between one and five times, and in another illustrative example the tape is applied and removed from the skin four times.

Factors such as the flexibility, softness, and composition of the adhesive tape used, the time the tape is allowed to adhere to the skin before it is removed, the force applied to the tape as it is applied to the skin, the prevalence of a gene product being analyzed, the disease status of the skin, and patient/patient variability are typically taken into account in deciding on a protocol useful for a particular tape stripping method in order to assure that sufficient nucleic acids are present in the epidermal sample. A tape stripped sample includes, but may not be limited to, tissues that are restricted to the surface of skin and preferentially recovers vellus hair follicles and cells lining sebaceous, eccrine, and sweat ducts (i.e. the adnexal structures associated with the stratum comeum and epidermis), as well as corneocytes. Tape stripping is stopped before viable epidermis is exposed by ceasing tape stripping before the tissue glistens. Therefore, the tape stripping method is considered a “noninvasive” method. Tape stripping sufficient to isolate an epidermal sample is tape stripping that is performed on the skin sufficient times to obtain an RNA sample, wherein the tape stripping is stopped before the tissue glistens (i.e. becomes shiny, appears “moistened” or reflective).

Certain aspects of the invention, which themselves are embodiments of the invention, are based in part on the discovery that non-polar, pliable adhesive tapes, especially plastic-based adhesive tapes, are more effective for obtaining nucleic acid samples from the skin than other types of adhesive tapes. Using non-polar, pliable adhesive tapes as few as 10 or less tape strippings and in certain examples as few as 4 or even 1 tape stripping can be used to obtain a nucleic acids that can be analyzed. The method can be used as part of various embodiments provided herein, to isolate an RNA sample from the epidermis of skin, for gene expression analysis.

Accordingly, provided herein is a method for isolating a nucleic acid molecule from an epidermal sample from skin, including applying an adhesive tape to a target area of the skin in a manner sufficient to isolate an epidermal sample adhering to the adhesive tape, wherein the epidermal sample includes nucleic acid molecules, such as miRNA, wherein the tape includes a non-polar polymer adhesive, and wherein the tape is pliable. A nucleic acid molecule is then isolated from the epidermal sample. In illustrative examples, the non-polar polymer adhesive is a rubber-based adhesive.

The rubber based adhesive can be, for example, a synthetic rubber-based adhesive. The rubber based adhesive in illustrative examples, has high peel, high shear, and high tack. For example, the rubber based adhesive can have a peak force tack that is at least 25%, 50%, or 100% greater than the peak force tack of an acrylic-based tape such as D-squame™. D-squame™ has been found to have a peak force of 2 Newtons, wherein peak force of the rubber based adhesive used for methods provided herein, can be 4 Newtons or greater. Furthermore, the rubber based adhesive can have adhesion that is greater than 2 times, 5 times, or 10 times that of acrylic based tape. For example, D-squame™ has been found to have adhesion of 0.0006 Newton meters, whereas the rubber based tape provided herein can have an adhesion of about 0.01 Newton meters using a texture analyzer. Furthermore, in certain illustrative examples, the adhesive used in the methods provided herein has higher peel, shear and tack than other rubber adhesives, especially those used for medical application and Duct tape.

In addition to having higher peel, shear, and tack, the rubber-based adhesive is more hydrophobic than acrylic adhesives. Furthermore, the rubber based adhesive in illustrative examples is inert to biomolecules and to chemicals used to isolate biomolecules, especially nucleic acids such as DNA and RNA. Finally, the adhesive can be relatively soft compared to other tapes such as D-squame™.

The rubber-based adhesive is on a support, typically a film, that makes the tape pliable and flexible. In certain aspects, the tape can be soft and pliable. “Pliable” tape is tape that is easily bent or shaped. “Soft and pliable” tape is tape that is easily bent or shaped and yields readily to pressure or weight. The film can be made of any of many possible polymers, provided that the tape is pliable and can be used with a rubber adhesive. The thickness can be varied provided that the tape remains pliable. For example, the tape can be 0.5 mil to 10 mil in thickness, 1.0 to 5.0 mil in thickness. In one example, the tape contains a rubber adhesive on a 3.0 mil polyurethane film. For example the film can be a polyurethane film such as skin harvesting tape (Product No. 90068) available from Adhesives Research, Inc (Glen Rock, Pa.).

The Examples illustrate tape stripping methods provided herein. Generally, before contacting a skin site with adhesive tape, a skin site to be stripped is cleaned, for example using an antiseptic cleanser such as alcohol. Next, tape is applied to a skin site with pressure. Pressure can be applied for a fraction of a second, but can be applied for between 1 second and 5 minutes, typically between 10 seconds and 45 seconds. In certain illustrative examples, tape is applied for 30 seconds for each tape stripping. It will be understood that the amount of pressure applied to a skin site and the length of time for stripping can be varied to identify ideal pressures and times for a particular application. Generally, pressure is applied by manually pressing down the adhesive tape on the skin, however, objects, such as blunt, rounded objects can be used to assist in applying the tape to the skin, especially for areas of the skin from which it is more difficult to obtain nucleic acid samples from skin, such as uninvolved skin of a subject afflicted with psoriasis.

Virtually any size and/or shape of adhesive tape and target skin site size and shape can be used and analyzed, respectively, by the methods of the present invention. For example, adhesive tape can be fabricated into circular discs of diameter between 10 millimeters and 100 millimeters, for example between 15 and 25 millimeters in diameter. The adhesive tape can have a surface area of between about 50 mm² and 1000 mm², between about 100 mm² to 500 mm² or about 250 mm².

The epidermis of the human skin comprises several distinct layers of skin tissue. The deepest layer is the stratum basalis layer, which consists of columnar cells. The overlying layer is the stratum spinosum, which is composed of polyhedral cells. Cells pushed up from the stratum spinosum are flattened and synthesize keratohyalin granules to form the stratum granulosum layer. As these cells move outward, they lose their nuclei, and the keratohyalin granules fuse and mingle with tonofibrils. This forms a clear layer called the stratum lucidum. The cells of the stratum lucidum are closely packed. As the cells move up from the stratum lucidum, they become compressed into many layers of opaque squamae. These cells are all flattened remnants of cells that have become completely filled with keratin and have lost all other internal structure, including nuclei. These squamae constitute the outer layer of the epidermis, the stratum comeum. At the bottom of the stratum corneum, the cells are closely compacted and adhere to each other strongly, but higher in the stratum they become loosely packed, and eventually flake away at the surface.

The skin sample obtained using the tape stripping method includes, epidermal cells including cells comprising adnexal structures. In certain illustrative examples, the sample includes predominantly epidermal cells, or even exclusively epidermal cells. The epidermis consists predominantly of keratinocytes (>90%), which differentiate from the basal layer, moving outward through various layers having decreasing levels of cellular organization, to become the cornified cells of the stratum corneum layer. Renewal of the epidermis occurs every 20-30 days in uninvolved skin. Other cell types present in the epidermis include melanocytes, Langerhans cells, and Merkel cells. As illustrated in the Examples herein, the tape stripping method of the present invention, is particularly effective at isolating epidermal samples.

Nucleic acids can be isolated from the lysed cells and cellular material by any number of means well known to those skilled in the art. For example, a number of commercial products available for isolating polynucleotides, including but not limited to, RNeasy™ (Qiagen, Valencia, Calif.) and TriReagent™ (Molecular Research Center, Inc, Cincinnati, Ohio) can be used. The isolated polynucleotides can then be tested or assayed for particular nucleic acid sequences, including a polynucleotide encoding a cytokine. Methods of recovering a target nucleic acid within a nucleic acid sample are well known in the art, and can include microarray analysis.

In another embodiment, provided herein is a non-invasive method for identifying a predictive skin marker for response to treatment for a disease, pathological or physiological state, including: applying an adhesive tape to the skin of a subject afflicted with the disease, pathological or physiological state at a first time point, in a manner sufficient to isolate an epidermal sample including nucleic acid molecules and treating the subject for the disease, pathological or physiological state. It is then determined whether the disease, pathological or physiological state has responded to the treatment, and if so, whether the level of miRNA in the sample is predictive of response to treatment. In another embodiment, the method may further include correlation of the level of miRNA to expression of a nucleic acid molecule in the epidermal sample, wherein expression is predictive of response to treatment.

Expression of a nucleic acid molecule in the epidermal sample is predictive of response to treatment if expression of the nucleic acid molecule at the first time point is different in subjects that respond to treatment compared to subjects that do not respond to treatment. It will be understood that a variety of statistical analysis can be performed to identify a statistically significant association between expression of the nucleic acid molecule and response of the subject to the treatment. For example, expression of the nucleic acid in certain examples is elevated, in subjects that will not respond to treatment. Furthermore, expression of the nucleic acid can predict a level of response to treatment, for example partial or temporary response to treatment versus a full response.

In another embodiment, provided herein is a non-invasive method for predicting response to treatment for a disease, pathological or physiological state, including applying an adhesive tape to the skin of a subject afflicted with the disease, pathological or physiological state in a manner sufficient to isolate an epidermal sample that includes nucleic acid molecules such as miRNA. Deviation between subject's miRNA level and the standard values is indicative of a disease, pathological or physiological state. In another embodiment, a target nucleic acid molecule is detected in the epidermal sample, whose expression is further indicative of a response to treatment, thereby predicting response to treatment for the disease, pathological or physiological state.

The disease for embodiments directed at identifying a predictive skin marker, or predicting response to treatment by detecting a predictive skin marker, also referred to in these embodiments as a target nucleic acid molecule, can be virtually any skin disease. For example, the skin disease can be psoriasis or dermatitis, such as irritant contact dermatitis or allergic contact dermatitis. In aspects where the disease is psoriasis, the treatment can be, for example, a topical treatment, phototherapy, a systemic medication, or a biologic. In other embodiments, the disease is any systemic disease having a direct manifestation in the skin. One such systemic disease is diabetes, which affects wound healing, microvasculature and nerve growth in the skin. Furthermore, there are data indicating that miRNA is involved in hair follicle morphogenesis (Yi et al. Nature Genetics 38: 358-362, 2006), the development, detection, and prognosis of certain cancers, such as melanoma, chronic lymphocytic lymphoma, prostate cancer, and non-small cell lung cancer, and even virus-host cell interactions. Accordingly, changes in quality (i.e. gain of function or loss of function mutations) or quantity of miRNA effect organ development and disease development.

Samples from a tissue can be isolated by any number of means well known in the art. Invasive methods for isolating a sample include the use of needles, for example during blood sampling, as well as biopsies of various tissues. Due to the invasive nature of these techniques there is an increased risk of mortality and morbidity. The methods and kits of the present invention use a non-invasive.sampling method to obtain a skin sample. In certain examples, these methods are used along with conventional methods, such as a skin biopsy, to provide additional information.

As mentioned above, tape-harvested cells appear to represent an enrichment of a sub-population of cells found in a shave biopsy. Accordingly, in certain aspects, in addition to a tape stripping method provided herein, a biopsy can be taken at the site of tape stripping, such as a psoriatic lesion site, or at another skin site. Nucleic acid molecules from the biopsy can be isolated and analyzed. Analysis of the biopsy data can be combined with analysis of data from a tape stripping method to provide additional information regarding the psoriatic lesion.

In certain aspects a nucleic acid molecule from uninvolved epidermal tissue is obtained by applying an adhesive tape to skin of the subject in a manner sufficient to isolate an epidermal sample adhering to the adhesive tape, wherein the epidermal sample includes nucleic acid molecules and wherein the skin is unaffected by a disease to be tested. Then a nucleic acid molecule is isolated and recovered from the epidermal sample of the unaffected skin.

In certain aspects, the uninvolved skin can be from the upper arm or the upper back, although the methods are in no way limited to such regions. These sites appear to provide relatively plentiful quantities of nucleic acid molecules using tape strippings. For example, tape stripping can be performed on uninvolved skin over the deltoid or upper back over the scapular spine and the periauricular region, e.g., mastoid process. Tape stripping generally involves the skin surface and therefore may preferentially recover vellus hair follicles and cells lining sebaceous, eccrine and sweat ducts (i.e. adnexal structures) as well as corneocytes (not predicted to contain RNA).

Certain embodiments provided herein, are based in part on the discovery that the expression of certain genes can be used to monitor response to therapy. Accordingly, in another embodiment, provided herein is a method for monitoring a response of a human subject to treatment for a disease, pathological or physiological state, including applying an adhesive tape to the skin of the subject being treated for the disease, pathological or physiological state at a first time point and at least a second time point, in a manner sufficient to isolate an epidermal sample adhering to the adhesive tape at the first time point and at the second time point. The epidermal sample includes a nucleic acid molecule, wherein a change in expression of the nucleic acid molecule between the first time point and the second time point is indicative of a change in the disease, pathological or physiological state.

In a related embodiment, provided herein is a method for detecting a response of a subject to treatment for a disease, pathological or physiological state, comprising: treating the subject for a skin disease, pathological skin state; applying an adhesive tape to the skin of the subject in a manner sufficient to isolate an epidermal sample, wherein the epidermal sample includes nucleic acid molecules such as miRNA; and detecting or recovering a target nucleic acid molecule in the sample comprising nucleic acid molecules. Expression of the target nucleic acid molecule is informative regarding the disease, pathological or physiological state. Therefore, the method identifies a response of the subject to treatment for the disease, pathological or physiological state.

The detection can be a qualitative detection of whether the target gene is expressed, but is typically a quantitative expression level determination. In another embodiment, the detection is determining a deviation of the level of miRNA from a control sample. The method can be performed both prior to treatment and after treatment. In one aspect, the method is performed after treatment, but before a change in disease, pathological or physiological state is observed visually.

Time points for the monitoring and response-to-treatment methods provided herein, can include any interval of time, ideally within 24 to 48 hours after treatment has begun, but are typically 1-2 weeks, and more typically at least 1 month apart. For certain embodiments, time points are 2 months, 3 months, 6 months, 1 year, or 2 years apart. Samples can be taken at any number of time points, including 2, 3, 4, 5, etc. time points. Comparison of expression analysis data from different time points can be performed using any of the known statistical methods for comparing data points to assess differences in the data, including time-based statistical methods such as control charting. The disease, pathological or physiological state can be identified in the time series, for example, by comparing expression levels to a cut-off value, or by comparing changes in expression levels to determine whether they exceed a cut-off change value, such as a percent change cut-off value. In certain aspects, the first time point is prior to treatment, for example, prior to administration of a therapeutic agent, and the second time point is after treatment.

The disease, pathological or physiological state can be virtually any skin disorder. For example, the skin disorder can be psoriasis, dermatitis, or a skin infection, an allergic reaction, hives, seborrhea, irritant contact dermatitis, allergic contact dermatitis, hidradenitis suppurative, allergic purpura. Pityriasis rosea, Dermatitis herpetiformis, erythema nodosum, erythema multiforme, lupus erythematosus, a bruise, actinic keratoses, keloid, lipoma, a sebaceous cyst, a skin tag, xanthelasma, melanoma, basal cell carcinoma, squamous cell carcinoma, or Kaposi's sarcoma. In one aspect the disease, pathological or physiological state is male pattern baldness, or age related changes in the skin.

The change in expression levels of at least one nucleic acid molecule can be an increase or decrease in expression. Furthermore, depending on the particular nucleic acid and the particular disease, pathological or physiological state, an increase or decrease can indicate a response to treatment, or a lack of response. For example, the nucleic acid can encode a protein such as CD2, TNF, or IFN, and a decrease in expression at the second time point as compared to the first time point is indicative of positive response to treatment for psoriasis. As another example, the method can detect a decrease in expression of TNF, IFN, IL-12B, NPF, or IL-23B, wherein a decrease in expression is indicative of response to treatment for psoriasis. As another example, the method detects expression of a keratin 10, keratin 16, or keratin 17 gene product, wherein an increase in expression is indicative of response to treatment for dermatitis, such as irritant dermatitis.

In other aspects of this method, a population of genes are detected. For these aspects, the method can be performed using a microarray.

Accordingly, the methods provided herein can be used to characterize the outer surface of virtually any animal. In certain aspects, the methods are used to characterize and/or otherwise analyze the outer surface of a body of a mammalian subject. For example, the methods can be used to tape strip rodents, such as mice, as well as, rabbits, or pigs. In illustrative examples, the methods are used to analyze human skin.

Certain embodiments of the invention relate,specifically to psoriasis and are based in part on the discovery that nucleic acid samples, for example RNA and miRNA samples, from the epidermis of the skin can be obtained from psoriatic lesions using tape stripping in psoriasis patients. Psoriasis is a chronic, genetic, noncontagious skin disorder that appears in many different forms and can affect any part of the body, including the nails and scalp. Psoriasis is categorized as mild, moderate, or severe, depending on the percentage of body surface involved and the impact on the patient's quality of life (QoL). Psoriasis may be one of several types: plaque psoriasis, pustular psoriasis, erythrodermic psoriasis, guttate psoriasis or inverse psoriasis. As indicated herein, methods for staging provided herein, assist in determining the severity and type of psoriasis.

Although psoriasis may affect any area of the body, it is most commonly found on the scalp, elbows, knees, hands, feet, and genitals. Plaque psoriasis, the most common type of the disease, is characterized by raised, thickened patches of red skin covered with silvery-white scales. Other types of psoriasis are characterized by different signs and symptoms. For example, pustular psoriasis is characterized by pus-like blisters, erythrodermic psoriasis is characterized by intense redness and swelling of a large part of the skin surface, guttate psoriasis is characterized by small, drop-like lesions, and inverse psoriasis is characterized by smooth red lesions in the folds of the skin. Method provided herein help to distinguish between the various types of psoriasis.

Although psoriasis may be almost unnoticeable in its early stages, subjects often report an itching and/or burning sensation as the disease progresses. In particular, plaque psoriasis usually begins with small red bumps on the skin that progress to bigger, scaly patches that may become itchy and uncomfortable. As the scales accumulate, pink to deep red plaques with a white crust of silvery scales appear on the skin surface.

The lesion suspected of being a psoriatic lesion can be the result of Koebner's phenomenon. In this phenomenon psoriatic lesions appear at the site of injury, infection or other skin problem. The lesion may mark the initial onset of psoriasis, or may be a new lesion in an existing case of psoriasis. In certain examples, the site of tape stripping can be on the fingernails or toenails, which are known sites of psoriasis that can be involved in psoriatic arthritis.

A “psoriasis skin marker” is a gene whose expression level is different between skin samples at the site of a psoriatic lesion and skin samples of uninvolved skin. Therefore, expression of a psoriasis skin marker is related to, or indicative of, psoriasis. As discussed herein, all of the psoriasis skin markers illustrated herein exhibit increased expression in psoriatic lesion versus non-psoriatic skin cell. Methods provided herein, for examples methods using microarrays to perform gene expression analysis using samples obtained from tape stripped skin, can be used to identify additional psoriasis markers. The expression of these psoriasis makers can increase or decrease in psoriatic lesions.

Many statistical techniques are known in the art, which can be used to determine whether a statistically significant difference in expression is observed at a 90% or preferably a 95% confidence level. In certain examples, a greater than 4 fold increase or decrease can be used as a cut-off value for identifying a psoriasis skin marker.

Accordingly, the present invention provides a non-invasive method for isolating or detecting a nucleic acid molecule from an epidermal sample of a psoriatic lesion of a human subject, including applying an adhesive tape to the psoriatic lesion of the subject in a manner sufficient to isolate an epidermal sample adhering to the adhesive tape. The epidermal sample includes a nucleic acid molecule that is then isolated and/or detected.

The nucleic acid for example can encode a protein such as CD2, TNFα, or IFNγ. Expression of these genes can be analyzed in psoriatic lesions. These embodiments, are useful for monitoring response to treatment for psoriasis; for determining a treatment that is likely most effective, for genetically characterizing psoriasis; for diagnosing psoriasis; and for identifying and analyzing nucleic acids that are predictive for response to a treatment for psoriasis. Changes in expression of these genes is shown in the Examples provided herein to be associated with psoriasis. For example, expression of TNFα and CD2 are elevated in most patients with psoriasis. Furthermore, in certain patients TNFα, CD2, and IFNγ are elevated. Accordingly, in certain aspects, expression of TNFα and CD2 is analyzed. In other aspects, expression of TNFα, CD2, and IFNγ are analyzed.

Methods of the present invention which isolate and/or detect a nucleic acid sample from an epidermal sample of a psoriatic lesion have utility not only in detecting and staging a psoriatic lesion, but also in diagnosing, and prognosing psoriasis as well as monitoring response of a psoriatic lesion to treatment. These methods can also be used to identify a predictive skin marker to identify a lesion and/or a patient, that will respond to treatment for psoriasis.

Skin samples obtained on adhesive films can be frozen before being analyzed using the methods of the present invention. Typically, this is performed by snap-freezing a sample, as illustrated in the Examples herein, using liquid nitrogen or dry ice.

One or more of the nucleic acid molecules in a sample provided herein, such as a as an epidermal sample, can be amplified before or after they are isolated and/or detected. The term “amplified” refers to the process of making multiple copies of the nucleic acid from a single nucleic acid molecule. The amplification of nucleic acid molecules can be carried out in vitro by biochemical processes known to those of skill in the art. The amplification agent can be any compound or system that will function to accomplish the synthesis of primer extension products, including enzymes. It will be recognized that various amplification methodologies can be utilized to increase the copy number of a target nucleic acid in the nucleic acid samples obtained using the methods provided herein, before and after detection. Suitable enzymes for this purpose include, for example, E. coli DNA polymerase I, Taq polymerase, Klenow fragment of E. coli DNA polymerase I, T4 DNA polymerase, other available DNA polymerases, T4 or T7 RNA polymerase, polymerase muteins, reverse transcriptase, ligase, and other enzymes, including heat-stable enzymes (i.e., those enzymes that perform primer extension after being subjected to temperatures sufficiently elevated to cause denaturation or those using an RNA polymerase promoter to make aRNA from a DNA template, i.e. linearly amplified aRNA).

Suitable enzymes will facilitate incorporation of nucleotides in the proper manner to form the primer extension products that are complementary to each nucleotide strand. Generally, the synthesis will be initiated at the 3′-end of each primer and proceed in the 5′-direction along the template strand, until synthesis terminates, producing molecules of different lengths. There can be amplification agents, however, that initiate synthesis at the 5′-end and proceed in the other direction, using the same process as described above. In any event, the method of the invention is not to be limited to the amplification methods described herein since it will be understood that virtually any amplification method can be used.

One method of in vitro amplification, which can be used according to this invention, is the polymerase chain reaction (PCR) described in U.S. Pat. Nos. 4,683,202 and 4,683,195. It will be understood that optimal conditions for a PCR reaction can be identified using known techniques. In one illustrative example, RNA is amplified using the MessageAmp™ aRNA kit (as disclosed in the Examples herein).

The primers for use in amplifying the polynucleotides of the invention can be prepared using any suitable method, such as conventional phosphotriester and phosphodiester methods or automated embodiments thereof so long as the primers are capable of hybridizing to the polynucleotides of interest. One method for synthesizing oligonucleotides on a modified solid support is described in U.S. Pat. No. 4,458,066. The exact length of primer will depend on many factors, including temperature, buffer, and nucleotide composition. The primer must prime the synthesis of extension products in the presence of the inducing agent for amplification.

Primers used according to the method of the invention are complementary to each strand of nucleotide sequence to be amplified. The term “complementary” means that the primers must hybridize with their respective strands under conditions, which allow the agent for polymerization to function. In other words, the primers that are complementary to the flanking sequences hybridize with the flanking sequences and permit amplification of the nucleotide sequence. The 3′ terminus of the primer that is extended can have perfect base paired complementarity with the complementary flanking strand, or can hybridize to the flanking sequences under high stringency conditions.

Upon isolation and optional amplification, expression of one or more genes is analyzed. Analyzing expression includes any qualitative or quantitative method for detecting expression of a gene, many of which are known in the art. Non-limiting methods for analyzing polynucleotides and polypeptides are discussed below. The methods of analyzing expression of the present invention can utilize a biochip, or other miniature high-throughput technology, for detecting expression of two or more genes.

The method of the present invention typically involve isolation of RNA, including messenger RNA (mRNA), or miRNA from a skin sample. The RNA may be single stranded or double stranded. Enzymes and conditions optimal for reverse transcribing the template to DNA well known in the art can be used. Alternatively, the RNA can be amplified to form amplified RNA. The RNA can be subjected to RNAse protection assays. A DNA-RNA hybrid that contains one strand of each can also be used. A mixture of polynucleotides can also be employed, or the polynucleotides produced in a previous amplification reaction, using the same or different primers can be so used. In certain examples, a nucleic acid to be analyzed is amplified after it is isolated. It is not necessary that the sequence to be amplified be present initially in a pure form; it may be a minor fraction of a complex mixture.

In addition, RNAse protection assays can be used if RNA is the polynucleotide to be detected in the method. In this procedure, a labeled antisense RNA probe is hybridized to the complementary polynucleotide in the sample. The remaining unhybridized single-stranded probe is degraded by ribonuclease treatment. The hybridized, double stranded probe is protected from RNAse digestion. After an appropriate time, the products of the digestion reaction are collected and analyzed on a gel (see for example Ausubel et al., CURRENT PROTOCOLS IN MOLECULAR BIOLOGY, section 4.7.1 (1987)). As used herein, “RNA probe” refers to a ribonucleotide capable of hybridizing to RNA in a sample of interest. Those skilled in the art will be able to identify and modify the RNAse protection assay specific to the polynucleotide to be measured, for example, probe specificity can be altered, hybridization temperatures, quantity of nucleic acid etc. Additionally, a number of commercial kits are available, for example, RiboQuant™ Multi-Probe RNAse Protection Assay System (Pharmingen, Inc., San Diego, Calif.).

In another embodiment, a nucleic acid in the sample may be analyzed by a blotting procedure, typically a Northern blot procedure. For blotting procedures polynucleotides are separated on a gel and then probed with a complementary polynucleotide to the sequence of interest. For example, RNA is separated on a gel transferred to nitrocellulose and probed with complementary DNA to one of the genes disclosed herein. The complementary probe may be labeled radioactively, chemically etc.

Detection of a nucleic acid can include size fractionating the nucleic acid. Methods of size fractionating nucleic acids are well known to those of skill in the art, such as by gel electrophoresis, including polyacrylamide gel electrophoresis (PAGE). For example, the gel may be a denaturing 7 M or 8 M urea-polyacrylamide-formamide gel. Size fractionating the nucleic acid may also be accomplished by chromatographic methods known to those of skill in the art.

The detection of nucleic acids can optionally be performed by using radioactively labeled probes. Any radioactive label can be employed which provides an adequate signal. Other labels include ligands, colored dyes, and fluorescent molecules, which can serve as a specific binding pair member for a labeled ligand, and the like. The labeled preparations are used to probe for a nucleic acid by the Southern or Northern hybridization techniques, for example. Nucleotides obtained from samples are transferred to filters that bind polynucleotides. After exposure to the labeled polynucleotide probe, which will hybridize to nucleotide fragments containing target nucleic acid sequences, the binding of the radioactive probe to target nucleic acid fragments is identified by autoradiography (see Genetic Engineering, 1 ed. Robert Williamson, Academic Press (1981), pp. 72-81). The particular hybridization technique is not essential to the invention. Hybridization techniques are well known or easily ascertained by one of ordinary skill in the art. As improvements are made in hybridization techniques, they can readily be applied in the method of the invention.

Probes according to the present invention and used in a method of the present invention selectively hybridize to a target gene. In preferred embodiments, the probes are spotted on a bioarray using methods known in the art. As used herein, the term “selective hybridization” or “selectively hybridize,” refers to hybridization under moderately stringent or highly stringent conditions such that a nucleotide sequence preferentially associates with a selected nucleotide sequence over unrelated nucleotide sequences to a large enough extent to be useful in detecting expression of a skin marker. It will be recognized that some amount of non-specific hybridization is unavoidable, but is acceptable provide that hybridization to a target nucleotide sequence is sufficiently selective such that it can be distinguished over the non-specific cross-hybridization, for example, at least about 2-fold more selective, generally at least about 3-fold more selective, usually at least about 5-fold more selective, and particularly at least about 10-fold more selective, as determined, for example, by an amount of labeled oligonucleotide that binds to target nucleic acid molecule as compared to a nucleic acid molecule other than the target molecule, particularly a substantially similar (i.e., homologous) nucleic acid molecule other than the target nucleic acid molecule.

Conditions that allow for selective hybridization can be determined empirically, or can be estimated based, for example, on the relative GC:AT content of the hybridizing oligonucleotide and the sequence to which it is to hybridize, the length of the hybridizing oligonucleotide, and the number, if any, of mismatches between the oligonucleotide and sequence to which it is to hybridize (see, for example, Sambrook et al., “Molecular Cloning: A laboratory manual (Cold Spring Harbor Laboratory Press 1989)). An example of progressively higher stringency conditions is as follows: 2×SSC/0.1% SDS at about room temperature (hybridization conditions); 0.2×SSC/0.1% SDS at about room temperature (low stringency conditions); 0.2×SSC/0.1% SDS at about 42EC (moderate stringency conditions); and 0.1×SSC at about 68EC (high stringency conditions). Washing can be carried out using only one of these conditions, e.g., high stringency conditions, or each of the conditions can be used, e.g., for 10-15 minutes each, in the order listed above, repeating any or all of the steps listed. However, as mentioned above, optimal conditions will vary, depending on the particular hybridization reaction involved, and can be determined empirically.

A method for detecting one or more genes can alternatively employ the detection of a polypeptide product of one of these genes. For example, polypeptide products of one of the genes disclosed herein as associated with psoriasis or irritated skin, can be analyzed. The levels of such gene products are indicative of psoriasis or a skin irritation when compared to a normal or standard polypeptide profiles in a similar tissue. Thus, the expression pattern of a gene disclosed herein as associated with psoriasis or irritant dermatitis, will vary depending upon the presence and stage of psoriasis or irritant dermatitis respectively.

In this regard, the sample, as described herein, can be used as a source to isolate polypeptides. For example, following skin stripping, using the methods described above, cells isolated from the stratum corneum can be lysed by any number of means, and polypeptides obtained from the cells. These polypeptides can then be quantified using methods known to those of skill in the art, for example by protein microarrays, or ELISA analysis.

In another embodiment, the present invention provides a method for obtaining gene expression data from amplified nucleic acids that compensates for variability in amplification reactions. In this method, relative expression of a target nucleic acid molecule and a control nucleic acid molecule is compared to obtain relevant expression data. Accordingly, in certain embodiments provided herein, a ΔCₜ (delta Cₜ) value is determined in order to identify gene expression changes. This value and method, although illustrated herein with respect to tape stripped skin samples, can be used to identify differential gene expression in any tissue. It is especially useful, where it is relatively difficult to obtain sufficient RNA from a control sample.

The Cₜ value is the experimentally determined number of amplification (e.g. PCR) cycles required to achieve a threshold signal level (statistically significant increase in signal level (e.g. fluorescence) over background) for mRNAₓ and a control mRNA (Gibson, Heid et al. 1996; Heid, Stevens et al. 1996). The Cₜ values are typically determined using a target nucleic acid (e.g. mRNAx) primer and probe set, and a control mRNA primer and probe set. A ΔCₜ value is calculated by calculating a difference in the number of amplification cycles required to reach a threshold signal level between the target nucleic acid molecule and the control nucleic acid molecule. A difference in the ΔCₜ value at a target area versus another area of a subject's skin, such as a normal area, or an unaffected area, is indicative of a change in gene expression of the target nucleic acid molecule at the target area (this difference in ΔCₜ values is commonly referred to as the ΔΔCₜ value).

Using this ΔCₜ value, altered expression is detected by comparing expression of the target nucleic acid molecule with expression of a control nucleic acid molecule. The Examples provided herein, illustrate that the ΔCₜ value, which is normally used to calculate a ΔΔCₜ value (and thus a calibrated fold-change), is itself useful for characterizing the physiologic state of the epidermis without reference to a calibration site. Example 2 provides the formula and related disclosure for calculating a ΔCₜ value. It is illustrated herein, that although the art teaches using a ΔΔCₜ value and not a ΔCₜ value for analyzing expression data, a ΔCₜ value is useful for this purpose, and provides the advantage that it is not necessary to obtain a nucleic acid sample from a control site, where it may be difficult to obtain sufficient nucleic acid molecules.

The utility of ΔCₜ values is predicated upon the consistency of the PCR reaction conditions and the use of identical probes between samples. Given these prerequisites, data in the Examples herein, support the potential for ΔCₜ values being diagnostic indicators.

Accordingly, provided herein is a method for detecting a change in gene expression, including: applying a first adhesive tape to a target area of skin and a second adhesive tape to an unaffected area of the skin, in a manner sufficient to isolate an epidermal sample adhering to the first adhesive tape and the second adhesive tape, wherein the epidermal samples comprise nucleic acid molecules; and for each of the target area sample and the normal area sample, amplifying a target nucleic acid molecule and a control nucleic acid molecule. For each of the target area sample and the normal area sample, a target nucleic acid molecule and a control nucleic acid molecule are amplified and identifying, and a ΔCₜ value by calculated by calculating a difference in the number of amplification cycles required to reach a threshold signal level between the target nucleic acid molecule and a control nucleic acid molecule, wherein a difference in the ΔCₜ value at the target area versus the normal area is indicative of a change in gene expression of the target nucleic acid molecule at the target area. The Cₜ values are typically determined in the same amplification experiment (e.g. using separate reaction wells on the same multi-well reaction plate) using similar reaction conditions to other reactions.

Accordingly to the tape stripping method provided herein, a first population of adhesive tapes can be applied to the target region, and a second population of adhesive tapes can be applied to a normal area of skin or an unaffected area of skin. For example, one, two, three, four, etc. separate tape strips can be applied to the target area of the skin and nucleic acids on the tape strips can be amplified together in a first reaction vessel. A different one, two, three, four, etc. separate tape strips can be applied to a normal area of the skin and nucleic acids on these tape strips can be amplified together in a second reaction vessel. In the first vessel and the second vessel, both the control nucleic acid and the target nucleic acid are amplified.

The target area for this embodiment, is typically an area of skin suspected containing diseased skin or skin in a pathological or physiological state. For example, the target area can include a psoriatic lesion or a region of skin with the characteristics of dermatitis.

In certain examples, the control nucleic acid molecule is expressed from a housekeeping gene. For example, the control nucleic aid molecule can encode β-actin, GAPDH, 18S rRNA, 28S rRNA, or tubulin. The adhesive tape is typically applied from about one to ten times, or between one and ten identical adhesive tapes are applied, as discussed herein related to the tape stripping method provided herein. Furthermore, a method according to this embodiment can utilize a microarray to detect a population of target nucleic acid molecules.

In another embodiment, the present invention provides a method for sampling an epidermal layer other than skin, using the tape stripping method provided herein. For example, the tape stripping method can be used to obtain a nucleic acid sample from an epidermal layer of the mouth, throat, or nose, or of an organ such as the liver, pancreas, kidney, intestines, stomach, bladder, brain, heart, or lungs, etc. by introducing a tape strip into a subject and applying it to a surface of the organ. The organ can be sampled within a body of the subject or after the organ is removed from the subject. Furthermore, the tape stripping method can be used to sample cells grown in vitro or organs reconstructed in vitro, for example for organ transplantation.

In another embodiment the invention provides a kit for isolation of a miRNA molecule from an epidermal sample. The epidermal sample may be from a psoriatic lesion or a target area of skin suspected of being inflamed or other area having a disease, pathological or physiological state. The kit can include an adhesive tape for performing methods provided herein. Accordingly, in one embodiment, provided herein is a kit, including a pliable adhesive tape made up at least in part, of a non-polar polymer. In certain aspects, the tape includes a rubber adhesive. In an illustrative example, the tape can be skin harvesting tape available (Product No. 90068) from Adhesives Research, Inc (Glen Rock, Pa.).

In addition to adhesive tape, the kit typically includes one or more detection reagents, for example probes and/or primers for amplification of, or hybridization to, a target nucleic acid sequence whose expression is related to a skin disease, pathological or physiological state. The probes or primers can be labeled with an enzymatic, florescent, or radionuclide label. For example, the probe can bind to a target nucleic acid molecule encoding a protein selected from CD2, TNFα, IFNγ, GAPDH, or Krt-16. Alternatively, the probe can be, for example, an antibody that binds the encoded protein. The probes can be spotted on a microarray which is provided in the kit.

The term “detectably labeled deoxyribonucleotide” refers to a deoxyribonucleotide that is associated with a detectable label for detecting the deoxyribonucleotide. For example, the detectable label may be a radiolabeled nucleotide or a small molecule covalently bound to the nucleotide where the small molecule is recognized by a well-characterized large molecule. Examples of these small molecules are biotin, which is bound by avidin, and thyroxin, which is bound by anti-thyroxin antibody. Other labels are known to those of ordinary skill in the art, including enzymatic, fluorescent compounds, chemiluminescent compounds, phosphorescent compounds, and bioluminescent compounds.

The kit can include one or more primer pairs, including a forward primer that selectively binds upstream of a gene whose expression is associated with psoriasis or irritant dermatitis, for example, on one strand, and a reverse primer, that selectively binds upstream of a gene involved in psoriasis or irritant dermatitis on a complementary strand. Primer pairs according to this aspect of the invention are typically useful for amplifying a polynucleotide that corresponds to a skin marker gene associated with psoriasis or contact dermatitis using amplification methods described herein.

A kit provided herein can also include a carrier means being compartmentalized to receive in close confinement one or more containers such as vials, tubes, and the like, each of the containers comprising one of the separate elements to be used in a method provided herein. If present, a second container may include, for example, a lysis buffer. The kit can alternatively include a computer-type chip on which the lysis of the cell will be achieved by means of an electric current.

Accordingly, kits provided herein can include an adhesive tape for tape stripping skin, such as rubber-based, pliable adhesive tape. The kits could include RNA isolation reagents and optionally primers and probes for genes whose expression is correlated with a skin disease, pathological skin state. Furthermore the kit could include primers and probes for control genes, such as housekeeping genes. The primers and probes for control genes can be used, for example, in determining Cₜ values for use in ΔCₜ calculations. The kits could also include instructions for performing tape strippings as well as for analyzing gene expression using ΔCₜ calculations.

The present invention is not to be limited in scope by the specific examples provided for below, which are intended as single illustrations of individual aspects of the invention and functionally equivalent methods and components are within the scope of the invention.

### EXAMPLE 1

### Recovery of Micro-RNA Molecules

The objective of this experiment was to recover micro-RNA (miRNA) from the surface of the skin with the use of adhesive tape.

Materials and Methods

Clinical protocol: Two subjects were tape stripped on the upper back at two adjacent sites. Each site was tape stripped with 4 individual tapes. Each tape was applied to the site with firm pressure and briskly rubbed with 15 circular motions covering the entire surface area of the tape. The tape was removed and the procedure repeated 3 more times, each time with a new tape. Used tapes were placed in a plastic envelope on ice until the procedure was competed and then stored at −80° C. until extraction.

Materials and reagents: Adhesive tape was purchased from Adhesives Research (Glen Rock, Pa.) in bulk rolls. These rolls were custom fabricated into small circular discs, 17 millimeters in diameter, by Diagnostic Laminations Engineering (Oceanside, Calif.). Human spleen total RNA was purchased from Ambion (catalogue # 7970; Austin, Tex.). RNeasy RNA extraction kit was purchased from Qiagen (Valencia, Calif.). Reverse transcriptase, PCR primers and probes, and TaqMan Universal Master Mix, which included all buffers and enzymes necessary for the amplification and fluorescent detection of specific cDNAs, were purchased from Applied Biosystems (Foster City, Calif.). MELT total nucleic acid isolation system and RecoverAll total nucleic acid isolation kit were purchased from Ambion (Austin, Tex.).

RNA isolation: RNA was extracted from tapes using either pressure cycling technology (PCT; Garrett, Tao et al. 2002; Schumacher, Manak et al. 2002) or MELT/Recoverall total nucleic acid system. Tapes were extracted in pairs by insertion into a PULSE™ tube (Pressure Biosciences, Gaithersburg, Md.) with 1.2 mls of buffer RLT (supplied in the Qiagen RNeasy kit). PULSE™ tubes were inserted into the PCT-NEP2017 pressure cycler and the sample was extracted using the following parameters: room temperature; 5 pressure cycles of 35 Kpsi with pressure held for 20 seconds at the top and bottom of each cycle. After pressure extraction the buffer was removed and used to process the remaining tapes used to strip that site; the buffer was then processed according to the standard Qiagen RNeasy protocol for the collection of larger RNAs (>200 nucleotides) by application to a purification column to which large RNA molecules (i.e. mRNAs) bind, while the column flow-through is saved for microRNA purification. The column flow-through, which contains miRNA separated from mRNA, is processed according to the Qiagen miRNA purification procedure (http://wwwl.qiagen.com/literature/protocols/pdf/RY20.pdf) to purify the microRNA. RNA from the 2 sites stripped on each subject was pooled to create a single sample from each subject.

MicroRNA isolation using MELT/RecoverAll total nucleic acid protocol: Tapes were extracted in pairs in a 2 ml eppendorf tube with 192 μl MELT buffer plus 8 μl of MELT cocktail and vortexed for 10 minutes at room temperature. The MELT lysates were transferred to the dispensed binding bead master mix after spinning down for 3 minutes at >10,000×g and washed with 300 μl of Wash Solution 1 and 2. RNAs including microRNAs were eluted in 100 μl of elution solution. MicroRNAs were further purified using the RecoverAll total nucleic acid protocol.

Reverse transcription and amplification/detection: 5 μl of RNA was reverse transcribed (RT) into cDNA with the TaqMan MicroRNA Reverse Transcription kit using the has-miR-16 RT primer in a final volume of 15 μl according to the manufacturer's directions. The reaction was diluted 6-fold with sterile, nuclease-free water for use in the subsequent amplification/detection reaction. For each specific miRNA detection assay, 3 replicate RT⁺ reactions and one RT⁻ (no reverse transcriptase; negative control) reaction were performed. Two amplification/detection reactions were done on each RT⁺ reaction to yield a total of 6 independent determinations of the threshold value (Cₜ; discussed below). Amplification and detection assays were performed using TaqMan MicroRNA Assay ABM 000008 has-miR-16 (Part # 4365746; Applied Biosystems, Foster City, Calif.) on an Applied Biosystems 7900HT Sequence Detection System as previously described (Wong, Tran et al. 2004). All RT reactions were amplified using 2 replicates and were negative (data not shown).

Quantitation of mRNA and recovery of miRNA: Experimental data is reported as the number of PCR cycles required to achieve a threshold fluorescence for a specific cDNA and is described as the “Cₜ” value (Gibson, Heid et al. 1996; Heid, Stevens et al. 1996; AppliedBiosystems 2001). Quantitation of total RNA mass was performed as previously described (Wong, Tran et al. 2004). Briefly, RNA mass recovered from tapes is determined by using quantitative RT-PCR with reference to a standard curve (C_{t, actin} vs. log[RNA]; AppliedBiosystems 2001) created from commercially purchased human spleen total RNA. The average of 6 replicate C_{t, actin} values was used to calculate the concentration of RNA in a sample with reference to the standard curve.

Results and Discussion

Two subjects were tape stripped on the upper back at two sites. The RNA was extracted from these tapes (two sites pooled to create one sample per subject) and assayed for total and miRNA. In our purification procedure larger mRNAs are separated from smaller miRNA, thus each sample is separated into mRNA and miRNA fractions. These fractions were assayed for total RNA and miRNA respectively. The results of these assays are shown in Table 1.

Table 1 shows that has-miR-16 miRNA is readily detectable in samples from both subjects. As a reference we also show the C_{t, actin} value in the mRNA fraction and the mass of RNA in that fraction. The fact that the miRNA Cₜ values are approximately 3 units lower than the C_{t, actin} values demonstrates that this specific miRNA is more easily detected than β-actin mRNA.

TABLE 1
Threshold values for miRNA and β-actin mRNA
detection and total RNA mass.

Cₜ^{a}

| Subject ID | has-miR-16 | β-actin | Total RNA^{b} |
|---|---|---|---|
| NRB | 20.87 ± 0.21 | 23.81 ± 0.18 | 116 ± 14 |
| PA | 20.59 ± 0.15 | 23.91 ± 0.10 | 109 ± 7.79 |

^{a}The threshold values for the miRNA miR-16 and β-actin mRNA.
^{b}Total RNA is reported in nanograms.

Four subjects were tape stripped on the mastoid process and the miRNA was extracted by MELT/RecoverAll total nucleic acid protocol and assayed for miRNAs. As shown in Table 2, several human miRNAs including has-mirR16, has-let7a, has-miR-20, has-miR-21, hasmir-17-5p, has-miR-130a, has-miR-191 and has-miR-200b are detectable in samples from four different subjects.

TABLE 2
Threshold value for miRNA detection

| | DTI00047B | | DTI00048B | | DTI00049B | | DTI00050B | |
|---|---|---|---|---|---|---|---|---|
| | mean | | mean | | mean | | mean | |
| Gene Name | Ct | stdev | Ct | stdev | Ct | stdev | Ct | stdev |
| has-miR-20 | 27.80 | 0.10 | 27.44 | 0.06 | 30.51 | 0.22 | 28.76 | 0.13 |
| has-miR-21 | 24.28 | 0.16 | 24.19 | 0.04 | 26.43 | 0.14 | 29.86 | 0.21 |
| has-miR-17-5p | 28.51 | 0.08 | 28.28 | 0.11 | 31.32 | 0.12 | 32.19 | 0.08 |
| has-miR-130a | 26.44 | 0.23 | 25.89 | 0.09 | 28.74 | 0.09 | 29.17 | 0.15 |
| has-miR-191 | 30.45 | 0.05 | 30.15 | 0.21 | 33.08 | 0.19 | 30.83 | 0.17 |
| has-miR-200b | 31.36 | 0.16 | 30.63 | 0.21 | 34.59 | 0.34 | 34.65 | 0.13 |
| has-let-7a | 25.53 | 0.12 | 24.57 | 0.13 | 27.97 | 0.21 | 32.25 | 0.67 |
| has-miR-16 | 26.40 | 0.15 | 25.85 | 0.12 | 29.04 | 0.12 | 23.81 | 0.03 |

In this work it was established that miRNAs can be isolated with tape from the surface of the skin. The ability to detect and recover miRNA in this fashion allows for miRNA profiling using tape as a collection method.

Just as skin diseases (e.g. psoriasis, atopic dermatitis) and afflictions (such as irritation, etc.) change the constellation of mRNAs in the skin, it is certain that miRNA populations will be similarly affected. Because of the role of miRNA in controlling mRNA expression, it is likely that some diseases and skin conditions may have miRNA profiles that do not change the mRNA profile (normal vs. diseased) but do change protein expression; thus it is possible that a change in miRNA profile will result in a changed physiological state by changing mRNA translation without affecting an mRNA profile.

However, it is equally likely that afflicted skin will have changes in both mRNA and miRNA profiles but that miRNA profiles may be easier to detect. In the data we presented (Table 1), the miRNA was easier to detect than β-actin mRNA, by extension some miRNA profiles may be more readily detected than mRNA profiles.

Some systemic diseases have direct manifestation in the skin. One example is diabetes which affects wound healing, microvasculature and nerve growth in the skin. These changes are likely accompanied by changes in mRNA and miRNA expression and thus tape stripping would not only be a non-invasive means of diagnosis of such diseases but also of monitoring the stages of such diseases by assaying changes of relevant miRNA in the skin. It is likely that such changes would precede clinical symptoms, thus giving health practitioners an early warning of these complications.

### EXAMPLE 2

### miRNA in Human Skin

Previously we had shown that micro RNA (miRNA) hsa-miR-16 could be recovered by tape stripping the skin. In this example, we extend this observation by testing for the presence of 34 distinct miRNA species.

Materials and Methods

Clinical protocol: 2 subjects were tape stripped on the upper back at 5 sites (4 tapes per site). The RNA collected from these sites was pooled into one sample for each subject.

PCR: Primers were supplied in the TaqMan MicroRNA Assays Human Panel—Early Access Kit (PN 4365409; Applied Biosystems). cDNA synthesis and amplification detection were performed as previously described. All reverse transcriptase minus controls were negative.

RNA from subject 1 was first tested for the presence of 153 miRNA supplied in the Human Assay Panel. Of the 157 miRNAs assayed, 129 were found to be present in subject 1's RNA sample.

In a second experiment, 34 miRNA from the human panel were chosen that have known homologues in mouse epidermis or hair follicle. These 34 miRNAs were then assayed in RNA samples from 2 subjects. The threshold values (Cₜ) for these assays are shown in Table 3.

The data in Table 3 clearly demonstrates that RNA recovered by tape stripping human skin by the methods described herein contains detectable levels of miRNA.

TABLE 3
Threshold values for 34 distinct miRNAs in human epidermis as detected in RNA recovered by tape
stripping.

| | Subject 1 | | Subject 2 | | | |
|---|---|---|---|---|---|---|
| miRNA^{a} | Mean^{b} Cₜ | SD^{b} Cₜ | Mean^{b} Cₜ | SD^{b} Cₜ | Epidermis^{c} | Hair Follicle^{c} |
| abm000175_hsa-let-7a | 24.95 | 0.16 | 27.69 | 0.08 | Yes | Yes |
| abm000008_hsa-miR-16 | 25.80 | 0.16 | 28.85 | 0.35 | Yes | Yes |
| abm000006_hsa-miR-15a | 28.32 | 0.04 | 29.26 | 0.52 | | Yes |
| abm000007_hsa-miR-15b | 27.66 | 0.07 | 30.85 | 0.46 | Yes | Yes |
| abm000010_hsa-miR-17-5p | 28.73 | 0.26 | 31.66 | 0.67 | Yes | Yes |
| abm000013_hsa-miR-20 | 28.28 | 0.43 | 30.17 | 0.71 | Yes | Yes |
| abm000014_hsa-miR-21 | 25.73 | 0.13 | 26.66 | 0.39 | Yes | Yes |
| abm000022_hsa-miR-27a | 27.98 | 0.29 | 30.19 | 0.43 | Yes | |
| abm000023_hsa-miR-27b | 26.47 | 0.33 | 27.92 | 0.31 | Yes | Yes |
| abm000029_hsa-miR-30b | 26.93 | 0.37 | 28.28 | 0.30 | Yes | |
| abm000036_hsa-miR-34a | 28.47 | 0.56 | 28.16 | 0.38 | Yes | |
| abm000039_hsa-miR-92 | 27.69 | 0.27 | 29.33 | 0.33 | Yes | Yes |
| abm000048_hsa-miR-103 | 27.75 | 0.28 | 28.92 | 0.75 | | Yes |
| abm000057_hsa-miR-125a | 29.11 | 0.09 | 29.70 | 0.17 | Yes | |
| abm000058_hsa-miR-125b | 27.42 | 0.28 | 28.33 | 0.28 | Yes | Yes |
| abm000059_hsa-miR-126 | 36.66 | 0.45 | 36.04 | 0.69 | | Yes |
| abm000060_hsa-miR-127 | 36.19 | 0.75 | 36.46 | 1.73 | | Yes |
| abm000064_hsa-miR-130a | 25.81 | 0.11 | 24.55 | 0.24 | Yes | Yes |
| abm000077_hsa-miR-141 | 27.06 | 0.18 | 28.43 | 0.45 | Yes | |
| abm000090_hsa-miR-152 | 30.56 | 0.19 | 31.37 | 0.23 | | Yes |
| abm000108_hsa-miR-191 | 30.20 | 0.27 | 32.90 | 0.20 | Yes | |
| abm000114_hsa-miR-199a | 32.59 | 0.40 | 33.05 | 0.62 | | Yes |
| abm000115_hsa-miR-199a* | 32.78 | 0.30 | 35.54 | 0.55 | | Yes |
| abm000116_hsa-miR-199b | 37.16 | 1.30 | ND | | | Yes |
| abm000118_hsa-miR-200a | 29.83 | 0.11 | 30.71 | 0.13 | Yes | |
| abm000119_hsa-miR-200b | 32.10 | 0.32 | 32.15 | 0.56 | Yes | |
| abm000120_hsa-miR-200c | 28.41 | 0.12 | 28.70 | 0.22 | Yes | |
| abm000121_hsa-miR-203 | 24.72 | 0.07 | 25.27 | 0.34 | Yes | |
| abm000123_hsa-miR-205 | 25.31 | 0.26 | 27.57 | 0.62 | Yes | |
| abm000129_hsa-miR-214 | 34.20 | 0.16 | 33.99 | 0.18 | | Yes |
| abm000176_hsa-let-7b | 26.27 | 0.09 | 27.05 | 0.14 | Yes | Yes |
| abm000177_hsa-let-7d | 26.13 | 0.29 | 27.75 | 0.21 | | Yes |
| abm000179_hsa-let-7g | 29.79 | 0.43 | 30.49 | 0.27 | Yes | Yes |
| abm000180_has-let-7i | 30.52 | 0.49 | 28.26 | 0.34 | Yes | Yes |

^{a}miRNA catalogue number (Applied Biosystems).
^{b}Mean calculated from 4 replicates; SD = standard deviation; ND indicated the miRNA was not detected,
^{c}Presence of homologue in mouse epidermis or hair follicle (Yi et al. Nature Genetics 38(3): 356-362 2006).

Although the invention has been described with reference to the above examples, it will be understood that modifications and variations are encompassed within the spirit and scope of the invention. Accordingly, the invention is limited only by the following claims.

What is claimed is:

1. A method for isolating miRNA molecules from an epidermal sample from skin, comprising:
a) applying an adhesive tape to a target area of the skin in a manner sufficient to isolate an epidermal sample adhering to the adhesive tape, wherein the epidermal sample comprises miRNA molecules; and
b) isolating miRNA molecules from the epidermal sample.

2. The method of claim 1, wherein the tape is a rubber-based tape.

3. The method of claim 2, wherein the tape comprises a rubber adhesive on a polyurethane film.

4. A method for isolating an miRNA sample from skin, comprising applying an adhesive tape to a target area of the skin in a manner sufficient to isolate an epidermal sample, wherein epidermal sample contains miRNA, wherein the tape comprises a rubber adhesive, and wherein the tape is pliable, thereby isolating an epidermal sample adhering to the adhesive tape.

5. The method of claim 4, wherein the tape is a rubber-based tape.

6. The method of claim 4, wherein the tape comprises a rubber adhesive on a polyurethane film.

7. The method of claims 1 or 4, wherein the tape is applied and removed from the skin about one to ten times.

8. The method of claims 1 or 4, wherein the tape is applied and removed from the skin about one to eight times.

9. The method of claims 1 or 4, wherein the tape is applied and removed from the skin about one to five times.

10. A method for identifying an miRNA profile indicative of a disease, pathological or physiological state of a human subject, the method comprising:
a) applying an adhesive tape to an area of skin in a manner sufficient to isolate an epidermal sample adhering to the adhesive tape, wherein the epidermal sample comprises miRNA molecules; and
b) determining miRNA levels from the sample, or an amplification product thereof, and comparing the levels with the miRNA levels of a control sample, wherein a difference in miRNA level is indicative of an miRNA profile of a disease, pathological or physiological state of a subject.

11. The method of claim 10, wherein the disease, pathological or physiological state is dermatitis.

12. The method of claim 10, wherein the disease, pathological or physiological state is psoriasis.

13. The method of claim 10, wherein the disease, pathological or physiological state is diabetes.

14. The method of claim 10, wherein the disease, pathological or physiological state is melanoma.

15. The method of claim 10, wherein the disease, pathological or physiological state is prostate cancer.

16. The method of claim 10, further comprising comparing the miRNA levels to expression levels of mRNA isolated from the same sample, thereby expression profiling the mRNA population in the sample.

17. A method for predicting response to treatment for a disease, pathological or physiological state, comprising:
a) applying an adhesive tape to an area of skin in a manner sufficient to isolate an epidermal sample adhering to the adhesive tape, wherein the epidermal sample comprises miRNA molecules; and
b) determining miRNA levels from the sample, or an amplification product thereof, and comparing the levels with the miRNA levels of an unaffected subject or sample, wherein a difference in miRNA level is indicative of a response to treatment of a disease, pathological or physiological state.

18. The method of claim 17, wherein the disease, pathological or physiological state is dermatitis.

19. The method of claim 17, wherein the disease, pathological or physiological state is psoriasis.

20. The method of claim 17, wherein the disease, pathological or physiological state is diabetes.

21. The method of claim 17, wherein the disease, pathological or physiological state is melanoma.

22. The method of claim 17, wherein the disease, pathological or physiological state is prostate cancer.
